(19) United States
(12) Patent Application Publication
(10) Pub. No.: US 2026/0215997 A1
(43) Pub. Date: Jul. 30, 2026

(54) MONITOR THAT ADVISES WHEN CPR WORSENS CIRCULATION

(71) Applicant: Stryker Corporation, Portage, MI (US)

(72) Inventor: Fred W. Chapman, Newcastle, WA (US)

(73) Assignee: Stryker Corporation, Portage, MI (US)

(21) Appl. No.: 19/464,210

(22) Filed: Jan. 29, 2026

Related U.S. Application Data
(60) Provisional application No. 63/750,978, filed on Jan. 29, 2025.

(51) Int. Cl. A61H 31/00; A61N 1/39

(52) U.S. Cl. CPC A61H 31/005; A61H 31/006; A61N 1/39044; A61H 2201/10; A61H 2201/1207; A61H 2201/5043; A61H 2230/065; A61H 2230/201; A61H 2230/255; A61H 2230/405

(57) ABSTRACT

Systems, devices, and methods for determining a treatment decision for a subject during cardiopulmonary resuscitation (CPR) are described herein. In an example method, a physiological parameter is detected during a first time period when treatment is administered to the subject and during a second time period when treatment is paused. The physiological parameter detected during treatment and the physiological parameter detected outside of treatment are analyzed. A treatment decision is identified based on analyzing the physiological parameter during treatment and outside of treatment. The example method can be implemented into a monitor-defibrillator or another portable medical device. Together, the methods can improve the treatment of subjects receiving CPR by reducing unnecessary treatment.

### CROSS-REFERENCE TO RELATED APPLICATION

This application claims priority to U.S. Provisional App. No. 63/750,978, which was filed on January 29, 2025 and is incorporated by reference herein in its entirety.

### BACKGROUND

Cardiopulmonary resuscitation (CPR) is an emergency medical procedure to restore blood circulation and oxygenation in a patient suffering from cardiac arrest. CPR includes repeatedly administering chest compressions to pump blood through the body. Chest compressions can be delivered manually or by mechanical chest compression devices (MCCDs). Chest compressions can circulate blood to vital organs and other tissues of the patient when the patient’s heart is not spontaneously pumping blood through the patient’s body. Chest compressions can be administered while the patient is transported to a clinical setting for further care. CPR is generally administered until the patient regains spontaneous circulation, shows other signs of consciousness, or can receive further treatment in a clinical environment. Monitoring physiological parameters of the patient during CPR can provide useful information about the efficacy of CPR, such as whether chest compressions are adequately circulating blood through the patient’s body.

### BRIEF DESCRIPTION OF THE DRAWINGS

FIG. 1 illustrates an example environment in which a treatment device is administering an emergency medical procedure to a subject experiencing cardiac arrest.

FIG. 2 illustrates an example process for determining a treatment decision for a subject.

FIG. 3 illustrates an example situation in which a patient may be negatively impacted by treatment.

FIG. 4 illustrates an example of an external defibrillator configured to perform various functions described herein.

FIG. 5 illustrates a chest compression device configured to perform various functions described herein.

### DETAILED DESCRIPTION

Implementations of the present disclosure are directed to specific improvements in the technical field of emergency medicine and patient monitoring. In particular, implementations of the present disclosure relate to monitoring a condition of a subject while they are undergoing an emergency medical procedure and determining whether a subject’s condition can be improved by discontinuing the emergency medical procedure. For instance, emergency medical personnel are trained to provide chest compressions to individuals experiencing medical emergencies, but in some cases, the chest compressions negatively impact their health. Various implementations of the present disclosure indicate when chest compressions are beneficial and when they may be harmful.

Various implementations described herein relate to systems, devices, and methods for analyzing a physiological parameter detected while a treatment is administered to the subject and while the treatment is paused. Various methods described herein compare the physiological parameter during treatment to the physiological parameter outside of treatment. Various methods described herein determine a time period of treatment administration based on identifying a treatment artifact in the physiological parameter. Various methods described herein determine a time period when treatment is paused based on identifying a lack of the treatment artifact in the physiological parameter. Various methods described herein compare the physiological parameter during treatment and the physiological parameter outside of treatment to a first threshold value and the second threshold value, respectively. Accordingly, the impact of the treatment on the condition of the subject can be determined. Determining the impact of the treatment on the subject’s condition can inform treatment decisions (e.g., to discontinue treatment, to continue treatment, to adjust a treatment parameter, etc.). The use of various implementations described herein can improve the efficacy of medical treatment and avoid adverse effects on the subject’s condition due to, for instance, unnecessary medical treatment.

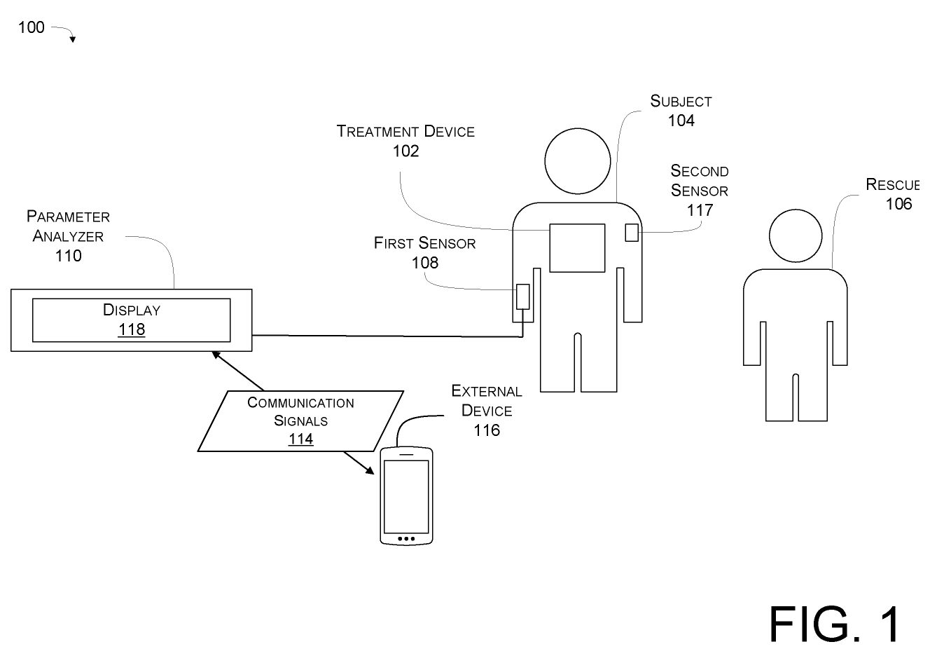

FIG. 1 illustrates an example environment 100 in which a treatment device 102 is administering an emergency medical procedure to a subject 104 experiencing cardiac arrest. During cardiac arrest, the heart of the subject 104 stops effectively pumping blood to a body of the subject 104. For instance, the subject 104 may have an arrhythmia (e.g., ventricular fibrillation (VF), ventricular tachycardia (VT), or the like) that prevents the heart from effectively pumping blood. That is, the subject 104 may have a condition that prevents the heart of the subject 104 from spontaneously circulating blood in the body of the subject 104. In some cases, the lack of spontaneous circulation causes the subject 104 to lose consciousness.

In various cases, the treatment device 102 is configured to administer a treatment (e.g., the emergency medical procedure) to restore blood circulation in the subject 104 until the subject 104 can receive further treatment in a clinical environment, such as a hospital, or until the subject is successfully resuscitated. The treatment, for instance, includes chest compressions, pace pulses, positive pressure ventilation, an electrical shock (e.g., a defibrillation shock), or any combination thereof. The treatment device 102 may include a mechanical chest compression device, an advanced life support monitor-defibrillator, a pacemaker, a ventilator, a pocket mask, or a bag-valve-mask resuscitator. In some cases, the treatment device 102 is a portable medical device configured to be operated outside of a clinical environment by the rescuer 106, such as an automated chest compression device. For example, the environment could be at the scene of a car crash, in an airport terminal, in a residence, or some other place in which the subject 104 is experiencing a sudden medical emergency. The rescuer 106 may bring the treatment device 102 to the subject 104 in response to the subject 104 losing consciousness. The treatment device 102, in some instances, administers the treatment to the subject 104 before or while the subject 104 is transferred to a clinical environment for further treatment.

In various examples, a rescuer 106 administers the treatment to the subject 104 or causes the treatment device 102 to administer the treatment to the subject 104. For example, the rescuer 106 places and/or operates the treatment device 102. The rescuer 106 may be, in some cases, a lay bystander, an untrained user, or the like. In some cases, the rescuer 106 is a medical professional, a trained user, or another individual assisting with the treatment of the subject 104.

According to some implementations, a first sensor 108 is configured to detect a physiological parameter of the subject 104. In some implementations, the first sensor 108 is configured to detect an airway parameter, such as a capnograph, an end-tidal gas parameter, a level of carbon dioxide (CO₂) (e.g., a CO₂ partial pressure, a CO₂ fraction, a CO₂ volume, or the like), a flow rate, an inspiratory or expiratory pressure, or another airway parameter. The first sensor 108 may include a blood gas analyzer, a capnography device, a manometer, a flow meter, or another sensor configured to detect an airway parameter. In various examples, the first sensor 108 is configured to detect the airway parameter during inhalation and/or exhalation of the subject 104.

The physiological parameter is, in some cases, indicative of a blood flow of the subject 104. In some implementations, the first sensor 108 is configured to detect a blood oxygenation, such as a cerebral oxygenation, net forward blood flow, continuous or pulsatile skin color, or a pulse of the subject 104. The first sensor 108 may include a cerebral oximeter, a pulse oximeter, or a blood gas analyzer. The first sensor 108 may include an emitter configured to output (e.g., emit) an incident beam (e.g., ultrasound and/or infrared light), and a receiver configured to detect a reflection or scatter of the incident beam. The Doppler shift between the incident beam and the reflection or scatter is, in various cases, indicative of the blood flow of the subject 104. In various instances, the first sensor 108 includes an imaging device, such as a CCD camera, a CMOS camera, a DSLR camera, or the like. The first sensor 108 may be configured to detect a color of a portion of the skin of the subject 104. The skin color of the subject 104, in various cases, is indicative of the blood flow of the subject 104. Accordingly, in some cases, the fluctuation of the skin color over time reflects the pulsatility of the blood flow of the subject 104. In some examples, the first sensor 108 is configured to detect a blood flow of the subject 104. For instance, the first sensor 108 is configured to detect a net forward blood flow in the carotid artery, the femoral artery, or the aorta of the subject 104. The first sensor 108 may include a transcutaneous blood gas monitor, transducer (e.g., an ultrasound transducer), an electromagnetic flowmeter, a pulse oximeter, an arterial doppler probe, a laser Doppler flowmetry device, a sphygmomanometer, a blood pressure measuring system, or another sensor configured to detect the blood flow of the subject 104.

As used herein, the terms “flow,” “flow parameters,” and their equivalents may refer to one or more physiological parameters indicative of a movement of a gas or fluid in a subject (e.g., blood through a blood vessel, carbon dioxide (CO₂) in an airway, etc.). The term “flow rate,” and its equivalents, may refer to a volume or mass of the gas or fluid that passes a boundary (e.g., a cross-section of a blood vessel) with respect to time. The terms “net flow,” “net flow volume,” and their equivalents, may refer to overall movement of a substance, such as blood, during a particular discrete time interval. For instance, a net flow may refer to a volume or mass of the gas or fluid that passes a boundary (e.g., a cross-section of a blood vessel) during a time interval, such as during a cardiac cycle. A net flow volume can be calculated by integrating a flow rate over the time interval.

In some cases, it may be beneficial to determine when the treatment of the subject 104 can be discontinued. For instance, the subject 104 may experience return of spontaneous circulation (ROSC) during treatment administration, and the heart of the subject 104 may begin spontaneously circulating blood through the body of the subject 104. If the treatment continues after the subject 104 experiences ROSC, the treatment may reduce the circulation in the subject 104. The fluctuations in thoracic pressure from chest compressions may disrupt the blood flow through the heart, emptying blood from the chambers of the heart before the heart squeezes to eject the blood forwards through the circulatory system. ROSC can be identified by analyzing one or more physiological parameters of the subject 104 during the administration of chest compressions or during chest compression pauses. For example, ROSC can be identified by an abrupt increase in airway CO₂ (e.g., end-tidal CO₂) or by identifying blood flow using an ultrasound probe (e.g., a Doppler ultrasound probe). In general, ROSC is confirmed during chest compression pauses, so that the movement of the body of the subject 104 does not result in artifact that can confound detected physiological parameters, However, it can be difficult for untrained users to efficiently detect the return of a pulse in emergency situations, especially if the subject 104 has a weak pulse. Moreover, chest compression pauses can be harmful if the subject 104 does not have ROSC, because pausing the treatment reduces blood flow to vital organs and tissues.

These and other problems can cause the rescuer 106 to discontinue the treatment too early, which can harm the subject 104. For example, if the subject 104 has pulseless electrical activity (PEA), in which the ECG of the subject 104 includes QRS complexes but the subject 104 lacks a pulse, the rescuer 106 may erroneously conclude that the subject 104 has ROSC and discontinue chest compressions before the heart of the subject 104 can adequately circulate blood through the body of the subject 104.

In addition, these and other problems can cause the rescuer 106 to continue the treatment for a longer period than is necessary, which can also harm the subject 104. In various examples in which the heart of the subject 104 has returned to spontaneously pumping blood through the body of the subject 104, chest compressions can reinitiate an arrhythmia (e.g., VF) in the subject 104. Thus, administering the treatment unnecessarily may negatively impact a condition (e.g., a state of health) of the subject 104 after the subject has regained spontaneous circulation.

These issues can be addressed, in some implementations, by comparing at least one physiological parameter of the subject 104 when treatment is being administered (also referred to as “the physiological parameter during treatment") to the physiological parameter of the subject 104 when treatment is paused (also referred to as “the physiological parameter outside of treatment”). In particular examples, when the subject 104 does not have spontaneous circulation, the physiological parameter may indicate greater blood flow in the subject 104 during administration of a treatment, compared to before or after administration of the treatment. For instance, an increase in end-tidal CO₂ of the subject 104 during treatment administration indicates effective chest compressions and compression-related restoration of blood flow. In various instances, when the subject 104 has spontaneous circulation, the physiological parameter may indicate a decrease in blood flow in the subject 104 during treatment. The decrease of end-tidal CO₂ of the subject 104 during treatment may indicate lower blood flow and compression-related adverse effects. In various examples, the physiological parameter outside of treatment may be measured at a particular time delay from when treatment is paused. For instance, various physiological parameters, such as end-tidal CO₂ in the airway, may reflect CO₂ in the alveoli 10 to 30 seconds after chest compressions are paused. The increase of end-tidal CO₂ after chest compressions are paused may indicate that the subject 104 is not benefitting from the chest compressions. Based on comparing the physiological parameter during treatment and the physiological parameter outside of treatment, it may be determined that the treatment of the subject 104 can be discontinued. In some examples, stopping the treatment of the subject 104 may avoid adversely affecting the condition of the subject 104.

According to various implementations, a parameter analyzer 110 is configured to determine a treatment decision of the subject 104 based on receiving signals indicative of the physiological parameter from the first sensor 108. The treatment decision may include discontinuing the treatment, pausing the treatment for a particular time period, continuing the treatment, reinitiating the treatment, or changing a parameter of the treatment. In some examples, the treatment includes chest compressions, and the parameter includes a frequency, a depth, a duration, or a position of the chest compressions on the torso of the subject 104. In some examples, the treatment includes pacing, and the parameter includes a pulse rate (e.g., frequency), a pulse width (e.g., duration), or an amplitude of a current of electrical stimulations (e.g., pacing pulses) delivered to the subject 104 via electrodes disposed on a chest of the subject 104. In various instances, the treatment parameter may include repositioning the patient and/or the treatment device 102. In some examples, the treatment decision may include a decision to administer one or more medications to the subject 104, such as epinephrine, atropine, naloxone, dopamine, or the like.

In various examples, the first sensor 108 is connected to a transceiver configured to transmit signals indicative of the physiological parameter to the parameter analyzer 110. The transceiver is configured to transmit and receive communication signals using one or more communication network(s). In some implementations, the transceiver is configured to transmit signals to the parameter analyzer 110 in a wired fashion and/or wirelessly. For example, the communication network(s) includes one or more wireless networks that include a 3GPP network, such as an LTE radio access network (RAN) (e.g., over one or more LTE bands), an NR RAN (e.g., over one or more NR bands), or a combination thereof. In some instances, the communication signals are electromagnetic (EM) signals, radio waves, or the like. In some implementations, the transceiver is configured to communicate with external devices by transmitting and/or receiving signals wirelessly. The external devices, for example, include at least one of a sensor (e.g., the first sensor 108), a medical device (e.g., the treatment device 102), a computing device (e.g., the parameter analyzer 110), a mobile device, or a server. Examples of wireless networks include WI-FI®, cellular networks, wireless local area networks (WLANs), and BLUETOOTH®. In various examples, the transceiver includes a network interface card (NIC), a network adapter, a local area network (LAN) adapter, or a physical, virtual, or logical address to connect to the various external devices and/or systems. In various examples, the transceiver includes any sort of wireless transceivers capable of engaging in wireless communication (e.g., RF communication). In some examples, the transceiver transmits radio waves to the parameter analyzer 110 via a cell tower. In some cases, the transceiver is connected to a wireless modem, such as a modem for engaging in WI-FI®, WIGIG®, WIMAX®, BLUETOOTH®, or infrared communication. In some examples, the transceiver is configured to transmit signals in a wired fashion, such as by using a cable that connects the transceiver to the parameter analyzer 110. The transceiver and the parameter analyzer 110, in some implementations, include ports configured to receive connectors attached to the cable. In various examples, the transceiver includes a NIC to transmit data over the cable. Examples of wired connections include USB, USB-C, mini-USB, micro-USB, serial ports, and custom cables, among other examples.

According to some implementations, the parameter analyzer 110 is connected to the first sensor 108. The parameter analyzer 110 may be part of a medical device, such as the treatment device 102, the first sensor 108, or another medical device configured to monitor or treat the subject (e.g., a monitor-defibrillator, an external defibrillator, or the like). In some implementations, the parameter analyzer 110 is part of a computing device (e.g., a mobile device, or the like). The parameter analyzer 110 may be implemented in hardware (e.g., one or more processors), software (e.g., instructions executed by the processor(s)), or a combination thereof.

The parameter analyzer 110 is configured to determine when treatment is being administered to the subject 104. For instance, the parameter analyzer 110 may determine a first time period in which treatment is being administered to the subject 104 (e.g., by the treatment device 102 or the rescuer 106) and a second time period in which the treatment is not being administered to the subject 104. In some examples, the parameter analyzer 110 determines the first time period based on identifying an artifact associated with the treatment (also referred to as a “treatment artifact”) in the physiological parameter during the first time period. The parameter analyzer 110 may determine the second time period based on identifying that the treatment artifact is absent in the physiological parameter during the second time period.

In some examples, the parameter analyzer 110 may be configured to receive communication signals from the treatment device 102 indicating when treatment is being administered to the subject 104 and/or when treatment is paused. In various instances, the parameter analyzer 110 is a monitor-defibrillator that is configured to monitor a transthoracic impedance of the subject 104. The parameter analyzer 110 may identify chest compressions administered to the subject 104 by identifying oscillations in the transthoracic impedance of the subject 104. In some examples, the parameter analyzer 110 may be configured to receive communication signals 114 from an external device 116 (e.g., an input device) indicating when the treatment is being administered to the subject 104 and/or when treatment is paused. For instance, the rescuer 106 may transmit, using the external device 116, communication signals 114 to the parameter analyzer 110 indicating that treatment of the subject 104 has been paused.

The parameter analyzer 110, in some examples, is configured to determine a treatment decision based on comparing the physiological parameter during treatment to the first physiological parameter outside of treatment. The physiological parameter outside of treatment may be measured, in various cases, less than 5 seconds, 5 seconds, 10 seconds, 20 seconds, 30 seconds, 40 seconds, 50 seconds, 60 seconds, or more than 60 seconds after the treatment is paused. In various instances, the parameter analyzer 110 determines whether the physiological parameter during treatment is greater than the physiological parameter outside of treatment. The parameter analyzer 110 may determine a difference between the physiological parameter during treatment and the physiological parameter outside of treatment. For instance, the parameter analyzer 110 may determine a difference between the skin color of the subject 104 during treatment and the skin color of the subject 104 outside of treatment. In some cases, the parameter analyzer 110 determines a fluctuation of the skin color of the subject 104 during treatment and a fluctuation of the skin color of the subject 104 outside of treatment. In various cases, the parameter analyzer 110 determines whether the difference is indicative of an improvement or a deterioration in the condition of the subject 104. For instance, the parameter analyzer 110 may determine that the magnitude of the fluctuation in skin color outside of treatment is greater than the magnitude of the fluctuation in skin color during treatment, and therefore that the condition of the subject 104 is deteriorating during treatment. In some cases, the parameter analyzer 110 compares the physiological parameter during treatment and the physiological parameter outside of treatment to a first threshold and a second threshold, respectively. The parameter analyzer 110 may determine a first comparison based on comparing the physiological parameter during treatment to the first threshold. The parameter analyzer 110 may determine a second comparison based on comparing the physiological parameter outside of treatment to the second threshold. The first threshold may be different than the second threshold. In some examples, the first threshold is the same as the second threshold. The first threshold and the second threshold may be determined based on clinical guidelines or reference ranges associated with the physiological parameter (e.g., a normal range, an abnormal range, a baseline range for the subject 104, etc.).

In some examples, the parameter analyzer 110 may analyze the first comparison and the second comparison to determine the treatment decision. For example, the first comparison may indicate that the physiological parameter during treatment is less than the first threshold, and the second comparison may indicate that the physiological parameter outside of treatment is greater than the second threshold. Accordingly, the parameter analyzer 110 may determine that the treatment should be reinitiated or continued. In some examples, the first comparison may indicate that the physiological parameter during treatment is greater than the first threshold, and the second comparison may indicate that the physiological parameter outside of treatment is less than the second threshold. Accordingly, the parameter analyzer 110 may determine that the treatment should be discontinued.

In various implementations, the parameter analyzer 110 is configured to determine a first metric and a second metric corresponding to the physiological parameter during treatment and the physiological parameter outside of treatment, respectively. The first metric and the second metric may be representative of circulation of blood in the subject 104. For instance, the first metric and the second metric may be representative of pulmonary blood flow in the subject 104. The parameter analyzer 110 may determine a partial pressure of CO₂ over a particular time (e.g., 5 seconds, 10 seconds, 15 seconds, 20 seconds, 25 seconds, 30 seconds, 40 seconds, etc.). In various instances, the parameter analyzer 110 may identify a maximum partial pressure of CO₂ over the particular time or determine an average of the partial pressure of CO₂ over the particular time. In some examples, the parameter analyzer 110 determines a CO₂ concentration over a particular time. In various instances, the first metric and the second metric correspond to a volume of CO₂ per minute. The parameter analyzer 110 may compare the first metric to the second metric. In some examples, the parameter analyzer 110 compares the first metric and the second metric to the first threshold and the second threshold, respectively. In some examples, the parameter analyzer 110 determines the treatment decision based on comparing the first metric and the second metric. For example, the parameter analyzer 110 may determine that the CO₂ volume per minute during treatment is greater than the CO₂ volume per minute outside of treatment. Accordingly, the parameter analyzer 110 may determine that treatment should be reinitiated.

In various implementations, a second sensor 117 is configured to detect an additional physiological parameter. The second sensor 117 may include any sensor configured to detect a physiological parameter described herein. The additional physiological parameter, in some cases, is indicative of the blood flow of the subject 104. The additional physiological parameter, in some cases, is indicative of an airway parameter of the subject 104. The additional physiological parameter may be different than the first physiological parameter. In various instances, the second sensor 117 is a different kind of sensor than the first sensor 108. For instance, the second sensor 117 may be configured to detect an electrocardiogram, a blood oxygenation, a tissue oxygenation, a cerebral tissue oxygenation, an arterial blood pressure, or the like. In some examples, the first sensor 108 and the second sensor 117 are configured to detect an airway parameter and a blood flow parameter of the subject 104. For instance, the first sensor 108 may be configured to detect a capnograph of the subject 104, and the second sensor 117 may be configured to detect a cerebral oxygenation of the subject 104. Additional examples of physiological parameters and sensors, as well as combinations of sensors, are described in U.S. Pat. No 12,004,870, which is incorporated herein by reference.

The parameter analyzer 110 may determine the condition of the subject 104 based on receiving a signal indicative of the additional physiological parameter from the second sensor 117. The condition, in various examples, includes ROSC, PEA, VF, VT, a cardiac arrhythmia, spontaneous breathing, or the like. In some cases, the parameter analyzer 110 may determine the condition of the subject 104 based on the additional physiological parameter detected during the second time period (e.g., outside of treatment). In some examples, the parameter analyzer 110 may determine the condition of the subject 104 based on the additional physiological parameter detected during the first time period (e.g., during treatment). For instance, the parameter analyzer 110 may filter out a treatment artifact in the additional physiological parameter. In some examples, the parameter analyzer 110 compares the additional physiological parameter during treatment to a first threshold and the additional physiological parameter outside of treatment to a second threshold. The first threshold and the second threshold, in various cases, may be the same or may be different. In various examples, the parameter analyzer 110 determines a difference between the additional physiological parameter during treatment and the additional physiological parameter outside of treatment. The parameter analyzer 110 may compare the difference associated with the physiological parameter and the difference associated with the additional physiological parameter. For instance, the parameter analyzer 110 may determine whether both the physiological parameter and the additional physiological parameter are indicative of the subject’s condition improving during or due to treatment. Utilizing both the physiological parameter and the additional physiological parameter may provide greater confidence in the treatment decision.

The parameter analyzer 110 may output, via a display 118, an indication of the treatment decision. The display 118, in some examples, includes a visual display (e.g., a screen, an indicator light, etc.). In some cases, the display 118 includes an audio speaker. In some examples, the parameter analyzer 110 may transmit signals indicative of the treatment decision (e.g., the communication signals 114) to the external device 116 or to a medical device (e.g., the treatment device 102, the first sensor 108, or another medical device). In various implementations, based on receiving the signals indicative of the treatment decision, the treatment device 102 may be configured to alter or discontinue the treatment. In some examples, the rescuer 106 or a medical professional may use the external device 116 to transmit signals indicative of the treatment decision to the treatment device 102. The treatment device 102 may be a chest compression device configured to increase a frequency of chest compressions based on receiving a signal from the parameter analyzer 110.

The parameter analyzer 110, in various examples, may analyze the physiological parameter after outputting the indication of the treatment decision. For instance, the parameter analyzer 110 may be configured to determine a third time period when the treatment is altered or discontinued. The parameter analyzer 110 may compare the physiological parameter detected during the third time period to the physiological parameter detected during the first time period and/or to the physiological parameter detected during the second time period. Based on analyzing the physiological parameter during the third time period, the parameter analyzer 110 may, for instance, determine whether changing the treatment is beneficial for the subject 104. For instance, the parameter analyzer 110 may identify that the treatment device 102 has stopped administering the treatment to the subject 104, and the parameter analyzer 110 may determine that the volume of CO₂ per minute during the third time period is less than the volume of CO₂ per minute during the first time period. Accordingly, the parameter analyzer 110 may determine that treatment should be reinitiated. In various examples, the parameter analyzer 110 outputs an indication of the physiological parameter during the third time period via the display 118, the external device 116, or another medical device. According to some cases, the parameter analyzer 110 may continue to analyze the physiological parameter. For instance, the parameter analyzer 110 may compare the physiological parameter during the first and second time periods to the physiological parameter during a third time period, during which treatment is administered to the subject 104, and during a fourth time period, during which treatment is not administered to the subject 104. In some examples, the parameter analyzer 110 may determine that a first difference in the physiological parameter between the first and second time periods is greater than a second difference in the physiological parameter between the third and fourth time periods. The parameter analyzer 110 may determine that the subject 104 is benefitting from the treatment.

In some examples, the parameter analyzer 110 analyzes the additional physiological parameter after outputting the indication of the treatment decision. The parameter analyzer 110 may compare the additional physiological parameter detected during the third time period to the additional physiological parameter detected during the first time period and/or to the additional physiological parameter detected during the second time period. The parameter analyzer 110 may output an indication of the additional physiological parameter during the third time period via the display 118, the external device 116, or another medical device. The parameter analyzer 110, in some cases, may continue to analyze the additional physiological parameter, as described above with reference to the physiological parameter.

In various cases, the parameter analyzer 110 is configured to analyze the physiological parameter and/or the additional physiological parameter at multiple time points during treatment. In various cases, the parameter analyzer 110 is configured to analyze the physiological parameter and/or the additional physiological parameter at multiple time points outside of treatment. Repeated analysis may reduce the risk of noise or other contamination in the signals detected by the first sensor 108 or the second sensor 117 leading to inaccurate treatment decisions by the parameter analyzer 110. Further, repeated analysis, in some cases, provides greater confidence in the condition of the subject 104 and, accordingly, the treatment decision determined by the parameter analyzer 110. For instance, the parameter analyzer 110 may analyze the physiological parameter every 5 seconds, every 10 seconds, every 15 seconds, every 20 seconds, every 25 seconds, every 30 seconds, or at a different time interval outside of treatment. In various examples, the parameter analyzer 110 may analyze the physiological parameter at a random pattern of time intervals outside of treatment.

In various implementations, the techniques described herein can be applied to data collected from the subject 104. For instance, the first sensor 108 may transmit, to the parameter analyzer 110, data indicative of the physiological parameter. The first sensor 108 may transmit the data indicative of the physiological parameter in real-time (e.g., while the first sensor 108 is detecting the physiological parameter of the subject 104) or post-event (e.g., after the first sensor 108 has completed detecting the physiological parameter, after the subject 104 is transferred to a clinical setting, etc.). The parameter analyzer 110 may analyze the physiological parameter in real-time or post-event. In various implementations, post-event analysis may be used for further care of the subject 104 or for research purposes (e.g., to improve determination of future treatment decisions).

In various implementations, the subject 104 may lose consciousness due to VF, and the rescuer 106 may operate a chest compression device (e.g., the treatment device 102) to administer chest compressions to the subject 104. The subject 104 may experience ROSC during the administration of chest compressions. In some examples, the subject 104 may experience refibrillation (e.g., a second initiation of VF) if treatment is continued unnecessarily after ROSC occurs. In some examples, the parameter analyzer 110 may determine that circulation in the subject 104 during treatment is lower than the circulation in the subject 104 during treatment pauses. Accordingly, the parameter analyzer 110 may output, to the rescuer 106, an alert to discontinue chest compressions, thus reducing the risk of refibrillation of the subject 104 and preventing chest compressions from reducing the effectiveness of the spontaneous heart beats.

In some implementations, the subject 104 may collapse due to an abnormally slow heart rate (e.g., bradycardia). The rescuer 106 may operate a portable medical device configured to provide external pacing to the subject 104. The treatment device 102 may, in some examples, be an advanced life support monitor-defibrillator. The treatment device 102 may provide electrical stimulations to a heart of the subject 104 at a particular pulse rate, pulse width, and current amplitude to increase the rate of muscular contractions of the heart of the subject 104. In some examples, the heart of subject 104 may not contract in response to the pacing pulses. For instance, the current amplitude of the pacing pulses may be insufficient to cause muscular contraction. In some examples, the electrodes may not be positioned or adhered to the subject 104 properly.

In some examples, the subject 104 may experience complications (e.g., arrhythmia, dyspnea, palpitations, burns, pain, etc.) if the treatment is continued unsuccessfully. The parameter analyzer 110 may determine that the administered pacing pulses are not facilitating sufficient blood flow to the body of the subject 104. For instance, the parameter analyzer 110 may analyze at least one physiological parameter of the subject 104 that is indicative of blood flow, such as blood oxygenation, cerebral oxygenation, blood pressure, blood velocity, pulse wave velocity, volumetric blood flow, or the like. The parameter analyzer 110 may analyze the at least one physiological parameter during administration of pacing pulses and during a pause in pacing pulses. In various examples, the parameter analyzer 110 may determine when pacing pulses are being administered to the subject 104 based on the presence of a pacing artifact in the at least one physiological parameter.

The parameter analyzer 110 may determine that the heart of the subject 104 is not contracting in response to the pacing pulses. Accordingly, the parameter analyzer 110 may determine a pacing parameter, such as a current of the pacing pulses, to facilitate muscular contractions of the heart of the subject 104. For instance, the parameter analyzer 110 may determine that a greater amplitude of current may facilitate muscular contractions of the heart, also referred to as mechanically capturing the heart. In various examples, the parameter analyzer 110 may cause a discharge circuit of the treatment device 102 to output the electrical stimulations at the rate via electrodes disposed on a chest of the subject 104. In some cases, the parameter analyzer 110 may output an instruction to the rescuer 106 to change the rate of the pacing pulses. In various examples, the parameter analyzer 110 determines that the condition of the subject 104 (e.g., the blood oxygenation) is not improving in response to changing the pacing parameter. The parameter analyzer 110 may output an instruction to the rescuer 106 to discontinue the administration of pacing pulses to the subject 104. In some cases, the parameter analyzer 110 causes the discharge circuit of the treatment device 102 to discontinue the administration of the electrical stimulations to the subject 104. Accordingly, the subject 104 may regain cardiac function without suffering from additional complications.

In various implementations, the subject 104 may be unable to breathe due a respiratory condition (e.g., pneumonia, COVID, chronic obstructive pulmonary disease (COPD), acute respiratory distress syndrome (ARDS), opioid drug overdose, etc.), a brain injury (e.g., a stroke, traumatic brain injury (TBI), intracranial hemorrhage, etc.), or a cardiac condition. The rescuer 106 may operate a mechanical ventilator or a bag-valve mask (e.g., the treatment device 102) to provide artificial ventilation to the subject 104. The mechanical ventilator may facilitate the movement of air in to and out of the lungs of the subject 104. The subject 104 may, during treatment, regain respiratory function and the ability to breathe naturally.

In some examples, the subject 104 may experience complications (e.g., barotrauma, lung injury, decreased ventilation volume, respiratory muscle weakness, etc.) if treatment is continued unnecessarily after respiratory function is regained. In some examples, the parameter analyzer 110 may determine that the subject 104 is breathing spontaneously or has regained sufficient respiratory function to breathe naturally. For instance, the parameter analyzer 110 may analyze at least one physiological parameter of the subject 104 that is indicative of respiration, such as a respiratory rate, an airway pressure, a transpulmonary pressure, a pleural pressure, an intrathoracic pressure, an esophageal pressure, a tidal volume, blood oxygenation, or the like.

The parameter analyzer 110 may analyze the at least one physiological parameter during one or more inspiratory phases and/or expiratory phases of positive pressure ventilation provided by the treatment device 102. In various cases, the parameter analyzer 110 analyzes the physiological parameter(s) during a pause in (e.g., the inspiratory phase and/or the expiratory phase) of positive pressure ventilation provided by the treatment device 102. The parameter analyzer 110 may compare the physiological parameter(s) during treatment and outside of treatment to determine whether the subject 104 is benefiting from the treatment provided by the treatment device 102. For instance, the parameter analyzer 110 may determine that a tidal volume or a change in transpulmonary pressure has increased since the treatment device 102 has initiated the positive-pressure ventilation. In various cases, the parameter analyzer 110 determines that the physiological parameter(s) indicate that the subject 104 is benefiting from treatment. For instance, the parameter analyzer 110 may determine that a blood oxygenation of the subject 104 increases during treatment and/or that a tidal volume (e.g., end-tidal CO₂) decreases during treatment.

Accordingly, the parameter analyzer 110 may output, to the rescuer 106, an indication to continue the administration of the positive-pressure ventilation to the subject 104 based on determining that the subject 104 is benefiting from the treatment. In various instances, the parameter analyzer 110 determines, based on analyzing the physiological parameter(s), that the subject 104 is inhaling spontaneously during the inspiratory phase and/or expiratory phase. In some cases, the parameter analyzer 110 is configured to identify spontaneous breathing by detecting a local maximum or a local minimum in a ventilation parameter (e.g., airway flow rate, partial pressure of CO2, partial pressure of O2, capnograph, etc.), wherein the local maximum or the local minimum is asynchronous from the inspiratory and/or expiratory phase of the positive pressure ventilation provided by the treatment device 102. In some examples, the parameter analyzer 110 may determine, based on analyzing the physiological parameter(s), that the spontaneous breathing of the subject 104 is asynchronous with the phase delivered by the treatment device 102. Accordingly, the parameter analyzer 110 may output, to the rescuer 106, an alert to discontinue the administration of the positive-pressure ventilation, thus avoiding lung injury in the subject 104.

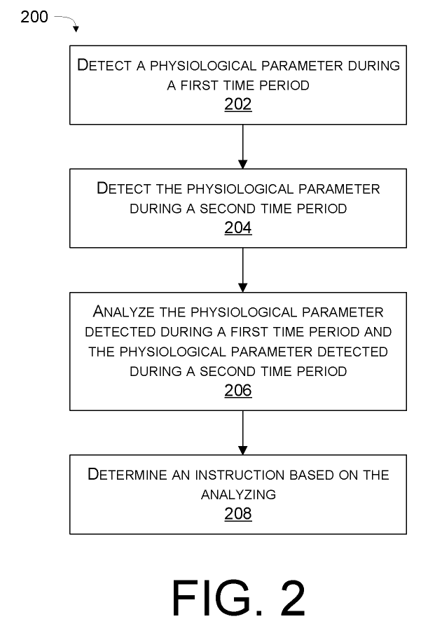

FIG. 2 illustrates an example method 200 for determining a treatment decision for a subject (e.g., the subject 104). According to some implementations, the method 200 is performed by an entity, such as a sensor (e.g., the first sensor 108 or the second sensor 117), medical device (e.g., the treatment device 102), computing device (e.g., the parameter analyzer 110), at least one processor, or a combination thereof.

At 202, the entity detects a physiological parameter of the subject during a first time period in which a treatment is being administered to the subject (e.g., the physiological parameter during treatment). The treatment may be administered by a treatment device or by a user (e.g., the rescuer 106).

At 204, the entity detects the physiological parameter of the subject during a second time period in which the treatment is not being administered to the subject (e.g., the physiological parameter outside of treatment). In various implementations, the first time period may be determined by identifying a treatment artifact (e.g., an artifact associated with the treatment) in the physiological parameter during the first time period. The second time period may be determined by identifying that the treatment artifact is not present in the physiological parameter during the second time period. In some examples, the entity may receive, from the treatment device or the user (e.g., via an input device), an indication of the first time period and the second time period.

At 206, the entity analyzes the physiological parameter during treatment and the physiological parameter outside of treatment. For instance, the entity may compare the physiological parameter during treatment to the physiological parameter outside of treatment. In some examples, the entity may determine a first comparison by comparing the physiological parameter during treatment to a first threshold. The entity may determine a second comparison by comparing the physiological parameter outside of treatment to a second threshold. The entity, in some examples, analyzed the first comparison to the second comparison. In various instances, the entity may determine a first metric indicative of the physiological parameter during treatment and a second metric indicative of physiological parameter outside of treatment. The first metric and the second metric, in some examples, correspond to a volume of CO₂ per minute.

At 208, the entity determines an instruction based on analyzing the physiological parameter during treatment and the physiological parameter outside of treatment. In some implementations, the entity determines the instruction based on analyzing the first metric and the second metric. In various instances, the entity determines the instruction based on comparing the first metric to the second metric. The instruction may include a treatment decision, such as an instruction to discontinue the treatment of the subject, to continue treatment of the subject, or to alter (e.g., change a parameter of) the treatment of the subject. In some cases, the entity may output, to the user, the instruction via a display. In some examples, the entity outputs the instruction to an external device or to the treatment device. In some examples, the treatment device may be configured to alter or discontinue the treatment of the subject in response to receiving, from the entity, a signal indicative of the instruction.

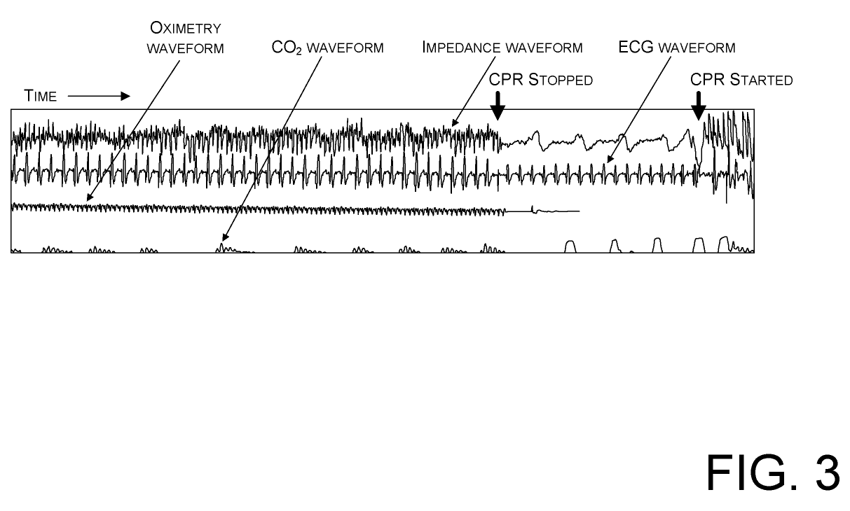

FIG. 3 illustrates an example situation in which a patient (e.g., the subject 104) is negatively impacted by treatment. Transthoracic impedance, ECG, pulse oximeter plethysmograph waveform, and airway CO₂ are illustrated from top to bottom. The patient’s heart is generating some blood flow, and CPR chest compressions are decreasing the blood flow. During the pause in CPR, indicated by flattening of impedance, the CO₂ waveforms become taller, indicating an increase in delivery of CO₂ to the lungs by the blood and, therefore, an increase in pulmonary blood flow. After CPR (e.g., chest compressions) is resumed, the CO₂ waveforms get progressively shorter, indicating a decrease in pulmonary blood flow. This is an example of a situation where the monitor (e.g., the parameter analyzer 110 described above with reference to FIG. 1) would advise the rescuer (e.g., the rescuer 106), and the rescuer should discontinue administering chest compressions and begin treating the patient as having profound hypotension instead of cardiac arrest.

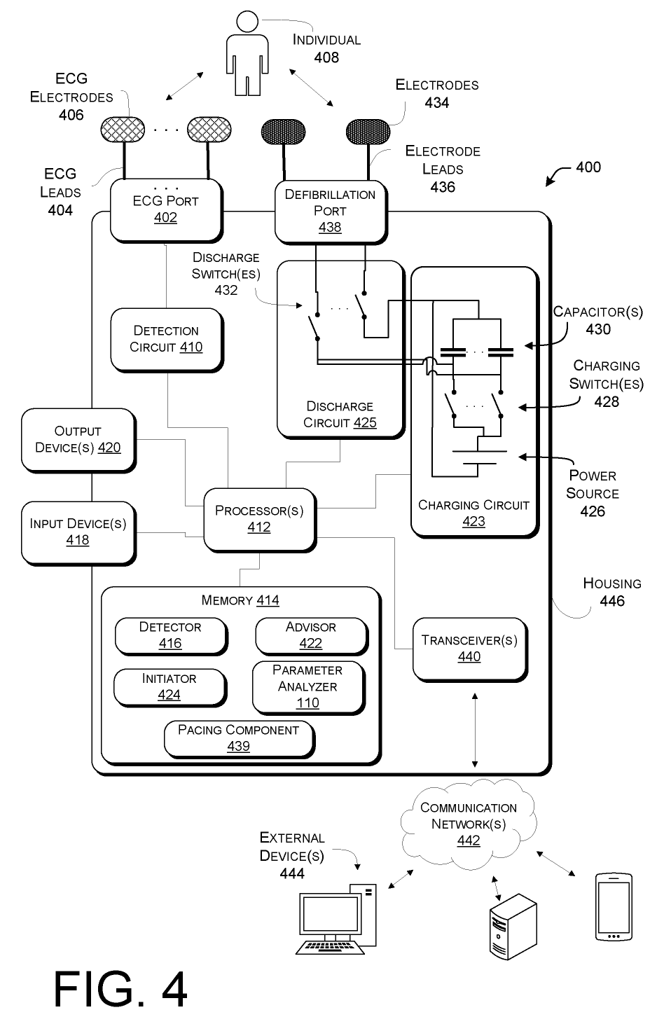

FIG. 4 illustrates an example of an external defibrillator 400 configured to perform various functions described herein. For example, the external defibrillator 400 includes the parameter analyzer 110 described above with reference to FIG. 1.

The external defibrillator 400 includes an electrocardiogram (ECG) port 402 connected to multiple ECG leads 404. In some cases, the ECG leads 404 are removeable from the ECG port 402. For instance, the ECG leads 404 are plugged into the ECG port 402. The ECG leads 404 are connected to ECG electrodes 406, respectively. In various implementations, the ECG electrodes 406 are disposed on different locations on an individual 408 (e.g., the subject 104). A detection circuit 410 is configured to detect relative voltages between the ECG electrodes 406. These voltages are indicative of the electrical activity of the heart of the individual 408.

In various implementations, the ECG electrodes 406 are in contact with the different locations on the skin of the individual 408. In some examples, a first one of the ECG electrodes 406 is placed on the skin between the heart and right arm of the individual 408, a second one of the ECG electrodes 406 is placed on the skin between the heart and left arm of the individual 408, and a third one of the ECG electrodes 406 is placed on the skin between the heart and a leg (either the left leg or the right leg) of the individual 408. In these examples, the detection circuit 410 is configured to measure the relative voltages between the first, second, and third ECG electrodes 406. Respective pairings of the ECG electrodes 406 are referred to as “leads,” and the voltages between the pairs of ECG electrodes 406 are known as “lead voltages.” In some examples, more than three ECG electrodes 406 are included, such that 5-lead or 12-lead ECG signals are detected by the detection circuit 410.

The detection circuit 410 includes at least one analog circuit, at least one digital circuit, or a combination thereof. The detection circuit 410 receives the analog electrical signals from the ECG electrodes 406, via the ECG port 402 and the ECG leads 404. In some cases, the detection circuit 410 includes one or more analog filters configured to filter noise and/or artifact from the electrical signals. The detection circuit 410 includes an analog-to-digital (ADC) in various examples. The detection circuit 410 generates a digital signal indicative of the analog electrical signals from the ECG electrodes 406. This digital signal can be referred to as an “ECG signal” or an “ECG.”

In some cases, the detection circuit 410 further detects an electrical impedance between at least one pair of the ECG electrodes 406. For example, the detection circuit 410 includes, or otherwise controls, a power source that applies a known voltage (or current) across a pair of the ECG electrodes 406 and detects a resultant current (or voltage) between the pair of the ECG electrodes 406. The impedance is generated based on the applied signal (voltage or current) and the resultant signal (current or voltage). In various cases, the impedance corresponds to respiration of the individual 408, chest compressions performed on the individual 408, and other physiological states of the individual 408. In various examples, the detection circuit 410 includes one or more analog filters configured to filter noise and/or artifact from the resultant signal. The detection circuit 410 generates a digital signal indicative of the impedance using an ADC. This digital signal can be referred to as an “impedance signal” or an “impedance.”

In various examples, the external defibrillator 400 is connected to a sensor (e.g., the first sensor 108 and/or the second sensor 117) configured to detect a blood circulation (e.g., a blood flow) of the individual 408. The detection circuit 410 may generate a digital signal indicative of the analog electrical signals from the sensor. This digital signal can be referred to as a “circulation signal” or a “circulation.”

The detection circuit 410 provides at least one of the ECG signal, the impedance signal, and the circulation signal to one or more processors 412 in the external defibrillator 400. In some implementations, the processor(s) 412 includes a central processing unit (CPU), a graphics processing unit (GPU), both CPU and GPU, or other processing unit or component known in the art. The processor(s) 412 may perform any of the functions described in relation to the parameter analyzer 110. For instance, the processor(s) 412 may determine, based on the circulation signal, whether the treatment is being administered to the individual 408. The processor(s) 412 may determine, based on the circulation signal, a treatment decision or a condition of the individual 408.

The processor(s) 412 is operably connected to memory 414. In various implementations, the memory 414 is volatile (such as random access memory (RAM)), non-volatile (such as read only memory (ROM), flash memory, etc.) or some combination of the two. The memory 414 stores instructions that, when executed by the processor(s) 412, causes the processor(s) 412 to perform various operations. In various examples, the memory 414 stores methods, threads, processes, applications, objects, modules, any other sort of executable instruction, or a combination thereof. In some cases, the memory 414 stores files, databases, or a combination thereof. In some examples, the memory 414 includes, but is not limited to, RAM, ROM, electrically erasable programmable read-only memory (EEPROM), flash memory, or any other memory technology. In some examples, the memory 414 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 412 and/or the external defibrillator 400. In some cases, the memory 414 at least temporarily stores at least one of the ECG signal, the impedance signal, and the circulation signal.

In some examples, the memory 414 includes the parameter analyzer 110. The parameter analyzer 110 may cause the processor(s) 412 to perform various functions described above with reference to FIG. 1. For instance, the parameter analyzer 110 may cause the processor(s) 412 to analyze one or more physiological parameters to determine whether a treatment is being administered to the individual 408. The parameter analyzer 110 may cause the processor(s) 412 to analyze the physiological parameter(s) and determine whether the treatment of the individual 408 should be discontinued.

In various examples, the memory 414 includes a detector 416, which causes the processor(s) 412 to determine, based on the ECG signal and/or the impedance signal, whether the individual 408 is exhibiting a particular heart rhythm. For instance, the processor(s) 412 determines whether the individual 408 is experiencing a shockable rhythm that is treatable by defibrillation. Examples of shockable rhythms include ventricular fibrillation (VF) and ventricular tachycardia (V-Tach). In some examples, the processor(s) 412 determines whether any of a variety of different rhythms (e.g., asystole, sinus rhythm, atrial fibrillation (AF), etc.) are present in the ECG signal.

The processor(s) 412 is operably connected to one or more input devices 418 and one or more output devices 420. Collectively, the input device(s) 418 and the output device(s) 420 function as an interface between a user and the defibrillator 400. The input device(s) 418 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 420 includes at least one of a display, a speaker, a haptic output device, a printer, or any combination thereof. In various examples, the processor(s) 412 causes a display (e.g., the display 118) among the output device(s) 420 to visually output a waveform of at least one of the ECG signal, the impedance signal, and the circulation signal. In some examples, the parameter analyzer 110 may cause the processor(s) 412 to provide, or cause the output device(s) 420 to provide, a visual indication to discontinue treatment of the individual 408. In some implementations, the input device(s) 418 includes one or more touch sensors, the output device(s) 420 includes a display screen, and the touch sensor(s) are integrated with the display screen. Thus, in some cases, the external defibrillator 400 includes a touchscreen configured to receive user input signal(s) and visually output physiological parameters, such as the ECG signal, the impedance signal, or the circulation signal.

In some examples, the memory 414 includes an advisor 422, which, when executed by the processor(s) 412, causes the processor(s) 412 to generate advice and/or control the output device(s) 420 to output the advice to a user (e.g., a rescuer). In some examples, the processor(s) 412 provides, or causes the output device(s) 420 to provide, an instruction to perform CPR on the individual 408. In some cases, the processor(s) 412 evaluates, based on the ECG signal, the impedance signal, the circulation signal, or other physiological parameters, CPR being performed on the individual 408 and causes the output device(s) 420 to provide feedback about the CPR in the instruction. According to some examples, the processor(s) 412, upon identifying that a shockable rhythm is present in the ECG signal, causes the output device(s) 420 to output an instruction and/or recommendation to administer a defibrillation shock to the individual 408.

The memory 414 also includes an initiator 424 which, when executed by the processor(s) 412, causes the processor(s) 412 to control other elements of the external defibrillator 400 in order to administer a defibrillation shock to the individual 408. In some examples, the processor(s) 412 executing the initiator 424 selectively causes the administration of the defibrillation shock based on determining that the individual 408 is exhibiting the shockable rhythm and/or based on an input from a user (received, e.g., by the input device(s) 418. In some cases, the processor(s) 412 causes the defibrillation shock to be output at a particular time, which is determined by the processor(s) 412 based on the ECG signal and/or the impedance signal.

The processor(s) 412 is operably connected to a charging circuit 423 and a discharge circuit 425. In various implementations, the charging circuit 423 includes a power source 426, one or more charging switches 428, and one or more capacitors 430. The power source 426 includes, for instance, a battery. The processor(s) 412 initiates a defibrillation shock by causing the power source 426 to charge at least one capacitor among the capacitor(s) 430. For example, the processor(s) 412 activates at least one of the charging switch(es) 428 in the charging circuit 423 to complete a first circuit connecting the power source 426 and the capacitor to be charged. Then, the processor(s) 412 causes the discharge circuit 425 to discharge energy stored in the charged capacitor across a pair of electrodes 434, which are in contact with the individual 408. For example, the processor(s) 412 deactivates the charging switch(es) 428 completing the first circuit between the capacitor(s) 430 and the power source 426, and activates one or more discharge switches 432 completing a second circuit connecting the charged capacitor 430 and at least a portion of the individual 408 disposed between electrodes 434.

The energy is discharged from the electrodes 434 in the form of a defibrillation shock. For example, the electrodes 434 are connected to the skin of the individual 408 and located at positions on different sides of the heart of the individual 408, such that the defibrillation shock is applied across the heart of the individual 408. The defibrillation shock, in various examples, depolarizes a significant number of heart cells in a short amount of time. The defibrillation shock, for example, interrupts the propagation of the shockable rhythm (e.g., VF or V-Tach) through the heart. In some examples, the defibrillation shock is 200 J or greater with a duration of about 0.015 seconds. In some cases, the defibrillation shock has a multiphasic (e.g., biphasic) waveform. The discharge switch(es) 432 are controlled by the processor(s) 412, for example. In various implementations, the electrodes 434 are connected to electrode leads 436. The electrode leads 436 are connected to a defibrillation port 438, in implementations. According to various examples, the electrode leads 436 are removable from the defibrillation port 438. For example, the defibrillation leads 436 are plugged into the defibrillation port 438.

In some implementations, the defibrillator 400 includes a pacing component 439 configured to cause the discharge circuit 425 to deliver electrical stimulation pulses to the electrodes 434 via the electrode leads 436. The pacing component 439 controls a current and/or a voltage of the electrical stimulation delivered to the electrodes 434. In some examples, the electrical stimulations have a current of 200 milliamperes (mA) or less. In some examples, the pacing component 439 delivers pulses of electrical stimulation at regular (or irregular) intervals to the electrodes in response to a pacing setting of the defibrillator 400 being activated. The pacing component 439 may also control a pulse width (e.g., a pulse duration) of the electrical stimulation.

In various implementations, the processor(s) 412 is operably connected to one or more transceivers 440 that transmit and/or receive data over one or more communication networks 442. For example, the transceiver(s) 440 includes a network interface card (NIC), a network adapter, a local area network (LAN) adapter, or a physical, virtual, or logical address to connect to the various external devices and/or systems. In various examples, the transceiver(s) 440 includes any sort of wireless transceivers capable of engaging in wireless communication (e.g., radio frequency (RF) communication). For example, the communication network(s) 442 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 440 includes other wireless modems, such as a modem for engaging in WI-FI®, WIGIG®, WIMAX®, BLUETOOTH®, or infrared communication over the communication network(s) 442.

The defibrillator 400 is configured to transmit and/or receive data (e.g., ECG data, impedance data, circulation data, data indicative of one or more detected heart rhythms of the individual 408, data indicative of one or more defibrillation shocks administered to the individual 408, etc.) with one or more external devices 444 via the communication network(s) 442. The external devices 444 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 442. In some examples, the external device(s) 444 is located remotely from the defibrillator 400, such as at a remote clinical environment (e.g., a hospital). According to various implementations, the processor(s) 412 causes the transceiver(s) 440 to transmit data to the external device(s) 444. In some cases, the transceiver(s) 440 receives data from the external device(s) 444 and the transceiver(s) 440 provide the received data to the processor(s) 412 for further analysis.

In various implementations, the external defibrillator 400 also includes a housing 446 that at least partially encloses other elements of the external defibrillator 400. For example, the housing 446 encloses the detection circuit 410, the processor(s) 412, the memory 414, the charging circuit 423, the transceiver(s) 440, or any combination thereof. In some cases, the input device(s) 418 and output device(s) 420 extend from an interior space at least partially surrounded by the housing 446 through a wall of the housing 446. In various examples, the housing 446 acts as a barrier to moisture, electrical interference, and/or dust, thereby protecting various components in the external defibrillator 400 from damage.

In some implementations, the external defibrillator 400 is an automated external defibrillator (AED) operated by an untrained user (e.g., a bystander, layperson, etc.) and can be operated in an automatic mode. In automatic mode, the processor(s) 412 automatically identifies a rhythm in the ECG signal, makes a decision whether to administer a defibrillation shock, charges the capacitor(s) 430, discharges the capacitor(s) 430, or any combination thereof. In some cases, the processor(s) 412 controls the output device(s) 420 to output (e.g., display) a simplified user interface to the untrained user. For example, the processor(s) 412 refrains from causing the output device(s) 420 to display a waveform of the ECG signal, the impedance signal, or the circulation signal to the untrained user, in order to simplify operation of the external defibrillator 400.

In some examples, the external defibrillator 400 is a monitor-defibrillator utilized by a trained user (e.g., a clinician, an emergency responder, etc.) and can be operated in a manual mode or the automatic mode. When the external defibrillator 400 operates in manual mode, the processor(s) 412 cause the output device(s) 420 to display a variety of information that may be relevant to the trained user, such as waveforms indicating the ECG data, impedance data, or the circulation data, notifications about detected heart rhythms or treatment decisions, and the like.

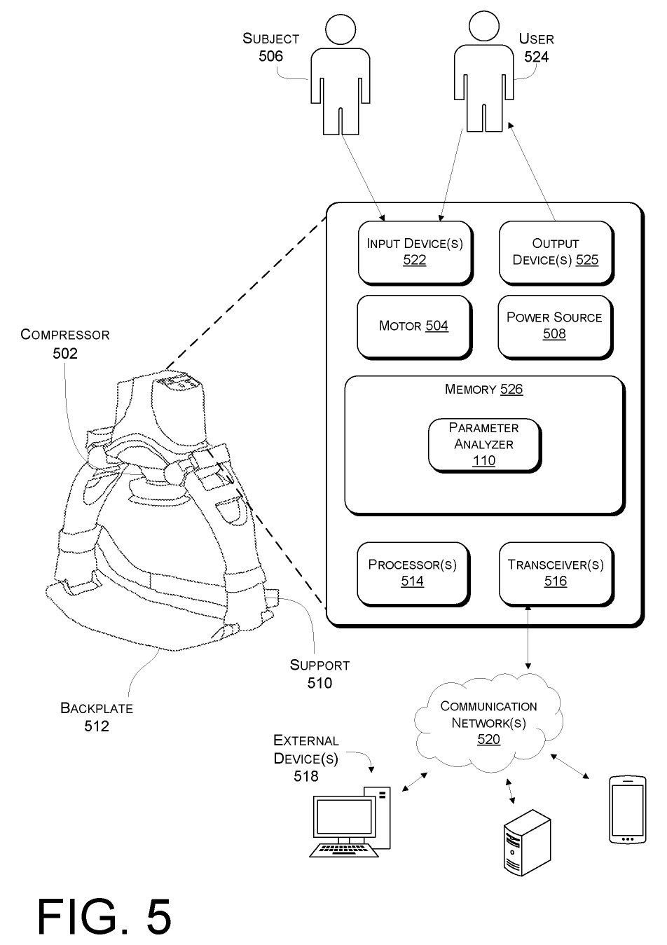

FIG. 5 illustrates a chest compression device 500 configured to perform various functions described herein. For example, the chest compression device 500 is the treatment device 102 described in FIG. 1.

In various implementations, the chest compression device 500 includes a compressor 502 that is operatively coupled to a motor 504. The compressor 502 physically administers a force to the chest of a subject 506 that compresses the chest of the subject 506. In some examples, the compressor 502 includes at least one piston that periodically moves between two positions (e.g., a compressed position and a release position) at a compression frequency. For example, when the piston is positioned on the chest of the subject 506, the piston compresses the chest when the piston is moved into the compressed position. A suction cup may be positioned on a tip of the piston, such that the suction cup contacts the chest of the subject 506 during operation. In various cases, the compressor 502 includes a band that periodically tightens to a first tension and loosens to a second tension at a compression frequency. For instance, when the band is disposed around the chest of the subject 506, the band compresses the chest when the band tightens.

The motor 504 is configured to convert electrical energy stored in a power source 508 into mechanical energy that moves and/or tightens the compressor 502, thereby causing the compressor 502 to administer the force to the chest of the subject 506. In various implementations, the power source 508 is portable. For instance, the power source 508 includes at least one rechargeable (e.g., lithium-ion) battery. In some cases, the power source 508 supplies electrical energy to one or more elements of the chest compression device 500 described herein.

In various cases, the chest compression device 500 includes a support 510 that is physically coupled to the compressor 502, such that the compressor 502 maintains a position relative to the subject 506 during operation. In some implementations, the support 510 is physically coupled to a backplate 512, cot, or other external structure with a fixed position relative to the subject 506. According to some cases, the support 510 is physically coupled to a portion of the subject 506, such as wrists of the subject 506.

The operation of the chest compression device 500 may be controlled by at least one processor 514. The processor(s) 514 may perform any of the functions described in relation to the parameter analyzer 110. In various implementations, the motor 504 is communicatively coupled to the processor(s) 514. Specifically, the processor(s) 514 is configured to output a control signal to the motor 504 that causes the motor 504 to actuate the compressor 502. For instance, the motor 504 causes the compressor 502 to administer the compressions to the subject 506 based on the control signal. In some cases, the control signal indicates one or more treatment parameters of the compressions. Examples of treatment parameters include a frequency, timing, depth, force, position, velocity, and acceleration of the compressor 502 administering the compressions. According to various cases, the control signal causes the motor 504 to cease compressions.

In various implementations, the chest compression device 500 includes at least one transceiver 516 configured to communicate with at least one external device 518 (e.g., the external device 116, the parameter analyzer 110) over one or more communication networks 520. Any communication network described herein can be included in the communication network(s) 520 illustrated in FIG. 4. The external device(s) 518, for example, includes at least one of a monitor-defibrillator, an AED, an ECMO device, a ventilation device, a patient monitor, a mobile phone, a server, or a computing device. In some implementations, the transceiver(s) 516 is configured to communicate with the external device(s) 518 by transmitting and/or receiving signals wirelessly. For example, the transceiver(s) 516 includes a NIC, a network adapter, a LAN adapter, or a physical, virtual, or logical address to connect to the various external devices and/or systems. In various examples, the transceiver(s) 516 includes any sort of wireless transceivers capable of engaging in wireless communication (e.g., RF communication). For example, the communication network(s) 520 includes one or more wireless networks that include a 3GPP network, such as an LTE RAN (e.g., over one or more LTE bands), an NR RAN (e.g., over one or more NR bands), or a combination thereof. In some cases, the transceiver(s) 516 includes other wireless modems, such as a modem for engaging in WI-FI®, WIGIG®, WIMAX®, BLUETOOTH®, or infrared communication over the communication network(s) 520. The signals, in various cases, encode data in the form of data packets, datagrams, or the like. In some cases, the signals are transmitted as compressions are being administered by the chest compression device 500 (e.g., for real-time feedback by the external device(s) 518), after compressions are administered by the chest compression device 500 (e.g., for post-event review at the external device 518), or a combination thereof.

In various cases, the processor(s) 514 generates the control signal based on data encoded in the signals received from the external device(s) 518. For instance, the signals include an instruction to initiate the compressions, and the processor(s) 514 instructs the motor 504 to begin actuating the compressor 502 in accordance with the signals.

In some cases, the chest compression device 500 includes at least one input device 522 (e.g., the external device 116). In various examples, the input device(s) 522 is configured to receive an input signal from a user 524, who may be a rescuer treating the subject 506 (e.g., the rescuer 106). Examples of the input device(s) 522 include, for instance, at a keypad, a cursor control, a touch-sensitive display, a voice input device (e.g., a microphone), a haptic feedback device (e.g., a gyroscope), or any combination thereof. In various implementations, the processor(s) 514 generate the control signal based on the input signal. For instance, the processor(s) 514 generate the control signal to adjust a frequency of the compressions based on the chest compression device 500 detecting a selection by the user 524 of a user interface element displayed on a touchscreen or detecting the user 524 pressing a button integrated with an external housing of the chest compression device 500.

According to some examples, the input device(s) 522 include one or more sensors (e.g., the first sensor 108, the second sensor 117). The sensor(s), for example, is configured to detect a physiological parameter of the subject 506. In some implementations, the sensor(s) is configured to detect a state parameter of the chest compression device 500, such as a position of the compressor 502 with respect to the subject 506 or the backplate 512, a force administered by the compressor 502 on the subject 506, a force administered onto the backplate 512 by the body of the subject 506 during a compression, or the like. According to some implementations, the signals transmitted by the transceiver(s) 516 indicate the physiological parameter(s) and/or the state parameter(s).

The chest compression device 500 further includes at least one output device 525, in various implementations. Examples of the output device(s) 525 include, for instance, least one of a display (e.g., a projector, an LED screen, etc.), a speaker, a haptic output device, a printer, or any combination thereof. In some implementations, the output device(s) 525 include a screen configured to display various parameters detected by and/or reported to the chest compression device 500, a charge level of the power source 508, a timer indicating a time since compressions were initiated or paused, and other relevant information.

The chest compression device 500 further includes memory 526. In various implementations, the memory 526 is volatile (such as random access memory (RAM)), non-volatile (such as read only memory (ROM), flash memory, etc.) or some combination of the two. The memory 526 stores instructions that, when executed by the processor(s) 514, causes the processor(s) 514 to perform various operations. In various examples, the memory 526 stores methods, threads, processes, applications, objects, modules, any other sort of executable instruction, or a combination thereof. In some cases, the memory 526 stores files, databases, or a combination thereof. In some examples, the memory 526 includes, but is not limited to, RAM, ROM, EEPROM, flash memory, or any other memory technology. In some examples, the memory 526 includes one or more of CD-ROMs, DVDs, CAM, or other optical storage, magnetic cassettes, magnetic tape, magnetic disk storage or other magnetic storage devices, or any other medium which can be used to store the desired information. In various cases, the memory 526 stores instructions, programs, threads, objects, data, or any combination thereof, that cause the processor(s) 514 to perform various functions. In various cases, the memory 526 stores one or more parameters that are detected by the chest compression device 500 and/or reported to the chest compression device 500.

In implementations of the present disclosure, the memory 526 includes the parameter analyzer 110. The parameter analyzer 110 may cause the processor(s) 514 to perform various functions described above with reference to FIG. 1. For instance, the parameter analyzer 110 may cause the processor(s) 514 to analyze one or more physiological parameters to determine whether a treatment is being administered to the subject 506. The parameter analyzer 110 may cause the processor(s) 514 to analyze the physiological parameter(s) and determine whether the treatment of the subject 506 should be discontinued. In various implementations, the parameter analyzer 110 causes the processor(s) 514 to output a control signal that causes the motor 504 to cease compressions. In some examples, the parameter analyzer 110 causes the processor(s) 514 to output a control signal that causes the motor 504 to change a treatment parameter of the compressions.

### EXAMPLE CLAUSES

1. A system, including: a sensor configured to detect, during a first time period and during a second time period, a flow of blood in a blood vessel of a subject; a display; and a processor configured to: determine that chest compressions are administered to the subject during the first time period; determine that the chest compressions are paused during the second time period; determine that the flow of blood during the second time period is greater than the flow of blood during the first time period; and in response to determining that the flow of blood during the second time period is greater than the flow of blood during the first time period, cause the display to output a recommendation to discontinue administering the chest compressions.

2. The system of clause 1, wherein the processor is configured to determine that the chest compressions are administered to the subject during the first time period by: identifying a chest compression artifact in data representative of the flow of blood through the blood vessel during the first time period, and wherein the processor is configured to determine that the chest compressions are paused during the second time period by: determining that the chest compression artifact is absent from the data representative of the flow of blood through the blood vessel during the second time period.

3. The system of clause 1 or 2, further including: an additional sensor configured to detect an additional physiological parameter indicative of blood circulation in the subject, wherein the processor is further configured to: determine a return of spontaneous circulation (ROSC) associated with the subject by analyzing the additional physiological parameter during the second time period; and cause the display to output an indication of the ROSC.

4. A medical device, including: a sensor configured to detect a physiological parameter indicative of blood circulation in a subject during a first time period in which a treatment is being administered to the subject and to detect the physiological parameter indicative of blood circulation in the subject during a second time period in which the treatment is paused; a processor configured to: determine a difference between the blood circulation during the first time period and the blood circulation during the second time period by comparing the physiological parameter detected during the first time period to the physiological parameter detected during the second time period; and determine a treatment parameter of the treatment in response to determining the difference.

5. The medical device of clause 4, wherein the sensor includes: an ultrasound transducer configured to: emit an ultrasound signal toward a blood vessel of the subject; and detect a reflection of the ultrasound signal from blood in the blood vessel of the subject, a Doppler shift between the ultrasound signal and the reflection of the ultrasound signal being indicative of a flow of blood through the blood vessel of the subject.

6. The medical device of clause 4 or 5, wherein the physiological parameter includes a level of carbon dioxide (CO₂) in an airway in the subject.

7. The medical device of any of clauses 4-6, wherein the physiological parameter includes a blood oxygenation of the subject.

8. The medical device of any of clauses 4-7, wherein the physiological parameter includes an electrocardiogram of the subject.

9. The medical device of any of clauses 4-8, wherein determining the treatment parameter includes: determining a third time period to continue administration of the treatment to the subject; or determining a fourth time period to pause administration of the treatment to the subject.

10. The medical device of any of clauses 4-9, wherein the treatment includes chest compressions, and wherein the treatment parameter includes at least one of a frequency, a depth, or a duration of chest compressions.

11. The medical device of clause 10, further including: a piston; and a motor configured to control the piston to administer a compressive force to a chest of the subject, wherein the processor is configured to cause the motor to control the compressive force administered to the subject in response to determining the treatment parameter.

12. The medical device of any of clauses 4-11, wherein the treatment parameter includes at least one of a rate, a width, or a current of pacing pulses.

13. The medical device of clause 12, further including: electrodes configured to deliver electrical stimulations to a patient via electrode leads; and a discharge circuit, wherein the processor is configured to cause the discharge circuit to provide the electrical stimulations via the electrodes in response to determining the treatment parameter.

14. The medical device of any of clauses 4-13, further including an output device configured to output an instruction to a user or an indication of the treatment parameter in response to determining the difference.

15. The medical device of clause 14, wherein the sensor is a first sensor, the physiological parameter is a first physiological parameter, and the medical device further includes: a second sensor configured to detect a second physiological parameter indicative of blood circulation in the subject during the second time period in which chest compressions are paused, the second physiological parameter is different from the first physiological parameter, wherein the processor is further configured to: determine a return of spontaneous circulation (ROSC) associated with the subject by analyzing the second physiological parameter during the second time period, and output, by the output device, an indication of ROSC.

16. The medical device of any of clauses 4-15, wherein the processor is configured to determine the difference between the blood circulation during the first time period and the blood circulation during the second time period by determining that the physiological parameter detected during the first time period is greater than the physiological parameter detected during the second time period, and wherein determining the treatment parameter includes determining a third time period to continue administration of the treatment to the subject.

17. The medical device of any of clauses 4-16, wherein the processor is configured to determine the difference between the blood circulation during the first time period and the blood circulation during the second time period by determining that the physiological parameter detected during the first time period is less than the physiological parameter detected during the second time period, and wherein determining the treatment parameter includes determining a third time period to pause administration of the treatment to the subject.

18. The medical device of any of clauses 4-17, wherein the processor is further configured to: determine that the treatment is administered during the first time period by identifying treatment artifacts exhibited in data representing the physiological parameter during the first time period.

19. The medical device of any of clauses 4-18, wherein the processor is further configured to: determine that the treatment is paused during the second time period by determining that a treatment artifact is absent from data representing the physiological parameter during the second time period.

20. A method, including: detecting a physiological parameter indicative of blood circulation in a subject during a first time period in which a treatment is being administered to the subject; detecting the physiological parameter indicative of blood circulation in the subject during a second time period in which the treatment is paused; determining a difference between the blood circulation during the first time period and the blood circulation during the second time period by comparing the physiological parameter detected during the first time period to the physiological parameter detected during the second time period; and in response to determining the difference, determining a treatment parameter of the treatment.

21. The method of clause 20, wherein detecting the physiological parameter indicative of blood circulation in the subject during the first time period in which chest compressions are being administered to the subject includes: emitting an ultrasound signal toward a blood vessel of the subject; and detecting a reflection of the ultrasound signal from blood in the blood vessel of the subject, a Doppler shift between the ultrasound signal and the reflection of the ultrasound signal being indicative of blood flow through the blood vessel of the subject.

22. The method of clause 20 or 21, wherein detecting the physiological parameter indicative of blood circulation in the subject during the first time period in which chest compressions are being administered to the subject includes determining a level of carbon dioxide (CO₂) in an airway in the subject.

23. The method of any of clauses 20-22, wherein detecting the physiological parameter indicative of blood circulation in the subject during the first time period in which chest compressions are being administered to the subject includes determining a level of blood oxygenation of the subject.

24. The method of any of clauses 20-23, further including: causing an output device to output an instruction to a user or an indication of the treatment parameter in response to determining the treatment parameter.

25. The method of any of clauses 20-24, further including: causing a motor to control a piston to administer a compressive force to a chest of the subject in response to determining the treatment parameter.

26. The method of any of clauses 20-25, further including: causing a discharge circuit to provide electrical stimulations to the subject via electrodes disposed on the subject in response to determining the treatment parameter.

27. The method of any of clauses 20-26, wherein: determining the difference of the blood circulation during the first time period and the blood circulation during the second time period includes determining that the physiological parameter detected during the first time period is less than the physiological parameter detected during the second time period, and determining the treatment parameter includes determining a third time period to pause administration of the treatment to the subject.

28. The method of any of clauses 20-27, wherein: determining the difference of the blood circulation during the first time period and the blood circulation during the second time period includes determining that the physiological parameter detected during the first time period is greater than the physiological parameter detected during the second time period, and determining the treatment parameter includes determining a third time period to continue administration of the treatment to the subject.

29. The method of any of clauses 20-28, further including: determining that the treatment is administered during the first time period by identifying treatment artifacts exhibited in data representing the physiological parameter during the first time period.

30. The method of any of clauses 20-29, further including: determining that the treatment is paused during the second time period by determining that a treatment artifact is absent from data representing the physiological parameter during the second time period.

31. A system, including: a sensor configured to detect, during a first time period and during a second time period, a flow of blood in a blood vessel of a subject; a display; and a processor configured to: determine that chest compressions are administered to the subject during the first time period; determine that the chest compressions are paused during the second time period; determine that the flow of blood during the first time period is less than a first threshold value; determine that the flow of blood during the second time period is greater than a second threshold value; and in response to determining that the flow of blood during the first time period is less than the first threshold value and determining that the flow of blood during the second time period is greater than the second threshold value, cause the display to output a recommendation to discontinue administering the chest compressions to the subject.

32. The system of clause 31, wherein the processor is configured to determine that the chest compressions are administered to the subject during the first time period by: identifying a chest compression artifact in data representative of the flow of blood through the blood vessel during the first time period, and wherein the processor is configured to determine that the chest compressions are paused during the second time period by: determining that data representative of the flow of blood through the blood vessel during the second time period omits the chest compression artifact.

33. The system of clause 31 or 32, wherein the flow of blood during the first time period is less than the flow of blood during the second time period.

34. A medical device, including: a sensor configured to detect a physiological parameter indicative of blood circulation in a subject during a first time period in which a treatment is being administered to the subject, and configured to detect the physiological parameter indicative of blood circulation in the subject during a second time period in which the treatment is paused; and a processor configured to: determine a first comparison between the physiological parameter during the first time period and a first threshold value indicative of blood circulation in the subject, and a second comparison between the physiological parameter during the second time period and a second threshold value indicative of blood circulation in the subject; and determine a treatment parameter of the treatment in response to determining the first comparison and the second comparison.

35. The medical device of clause 34, wherein the sensor includes: an ultrasound transducer configured to: emit an ultrasound signal toward a blood vessel of the subject; and detect a reflection of the ultrasound signal from blood in the blood vessel of the subject, a Doppler shift between the ultrasound signal and the reflection of the ultrasound signal being indicative of a flow of blood through the blood vessel of the subject.

36. The medical device of clause 34 or 35, wherein the physiological parameter includes a level of carbon dioxide (CO₂) in an airway in the subject.

37. The medical device of any of clauses 34-36, wherein the physiological parameter includes a blood oxygenation of the subject.

38. The medical device of any of clauses 34-37, wherein the physiological parameter includes an electrocardiogram of the subject.

39. The medical device of any of clauses 34-38, further including an output device configured to output an instruction to a user or an indication of the treatment parameter in response to determining the first comparison and the second comparison.

40. The medical device of clause 39, wherein the sensor is a first sensor, the physiological parameter is a first physiological parameter, and the medical device further includes: a second sensor configured to detect a second physiological parameter indicative of blood circulation in the subject during the second time period in which the treatment is paused, the second physiological parameter is different from the first physiological parameter, wherein the processor is further configured to: determine a return of spontaneous circulation (ROSC) associated with the subject by analyzing the second physiological parameter during the second time period, and output, by the output device, an indication of the ROSC.

41. The medical device of any of clauses 34-40, wherein the processor is configured to: determine the first comparison by determining that the physiological parameter during the first time period is less than the first threshold value, and determine the second comparison by determining that the physiological parameter during the second time period is greater than the second threshold value, wherein determining the treatment parameter includes determining a third time period to continue administration of the treatment to the subject.

42. The medical device of any of clauses 34-41, wherein the processor is configured to: determine the first comparison by determining that the physiological parameter during the first time period is greater than the first threshold value, and determine the second comparison by determining that the physiological parameter during the second time period is less than the second threshold value, and wherein determining the treatment parameter includes determining a third time period to pause administration of the treatment to the subject.

43. The medical device of any of clauses 34-42, wherein the processor is further configured to: determine that the treatment is administered during the first time period by identifying a treatment artifact exhibited in data representing the physiological parameter during the first time period.

44. The medical device of any of clauses 34-43, wherein the processor is further configured to: determine that the treatment is paused during the second time period by determining that a treatment artifact is absent from data representing the physiological parameter during the second time period.

45. The medical device of any of clauses 34-44, wherein the treatment includes chest compressions, and wherein the treatment parameter includes at least one of a frequency, a depth, or a duration of chest compressions.

46. The medical device of clause 45, further including: a piston; and a motor configured to control the piston to administer a compressive force to a chest of the subject, wherein the processor is configured to cause the motor to control the compressive force administered to the subject in response to determining the treatment parameter.

47. The medical device of any of clauses 34-46, wherein the treatment parameter includes at least one of a rate, a width, or a current of pacing pulses.

48. The medical device of clause 47, further including: electrodes configured to deliver electrical stimulations to a patient via electrode leads; and a discharge circuit, wherein the processor is configured to cause the discharge circuit to provide the electrical stimulations via the electrodes in response to determining the treatment parameter.

49. A method, including: detecting a physiological parameter indicative of blood circulation in a subject during a first time period in which a treatment is being administered to the subject; detecting the physiological parameter indicative of blood circulation in the subject during a second time period in which the treatment is paused; determining a first comparison between the physiological parameter during the first time period and a first threshold value indicative of blood circulation in the subject, and a second comparison between the physiological parameter during the second time period and a second threshold value indicative of blood circulation in the subject; and in response to determining the first comparison and the second comparison, determining a treatment parameter of the treatment.

50. The method of clause 49, wherein detecting the physiological parameter indicative of blood circulation in the subject during the first time period in which the treatment is being administered to the subject includes: emitting an ultrasound signal toward a blood vessel of the subject; and detecting a reflection of the ultrasound signal from blood in the blood vessel of the subject, a Doppler shift between the ultrasound signal and the reflection of the ultrasound signal being indicative of blood flow through the blood vessel of the subject.

51. The method of clause 49 or 50, wherein detecting the physiological parameter indicative of blood circulation in the subject during the first time period in which the treatment is being administered to the subject includes determining a level of carbon dioxide (CO₂) in an airway in the subject.

52. The method of any of clauses 49-51, wherein detecting the physiological parameter indicative of blood circulation in the subject during the first time period in which the treatment is being administered to the subject includes determining a level of blood oxygenation of the subject.

53. The method of any of clauses 49-52, wherein detecting the physiological parameter indicative of blood circulation in the subject during the first time period in which the treatment is being administered to the subject includes determining an electrocardiogram of the subject.

54. The method of any of clauses 49-53, wherein: determining the first comparison includes determining that the physiological parameter during the first time period is greater than the first threshold value, determining the second comparison includes determining that the physiological parameter during the second time period is less than the second threshold value, and determining the treatment parameter includes determining a third time period to continue administration of the treatment to the subject.

55. The method of any of clauses 49-54, wherein: determining the first comparison includes determining that the physiological parameter during the first time period is less than the first threshold value, determining the second comparison includes determining that the physiological parameter during the second time period is greater than the second threshold value, and determining the treatment parameter includes determining a third time period to pause administration of the treatment to the subject during a third time period.

56. The method of any of clauses 49-55, further including: determining that the treatment is administered during the first time period by identifying treatment artifacts exhibited in data representing the physiological parameter during the first time period.

57. The method of any of clauses 49-56, further including: determining that the treatment is paused during the second time period by determining that a treatment artifact is absent from data representing the physiological parameter during the second time period.

58. The method of any of clauses 49-57, further including: causing an output device to output an instruction to a user or an indication of the treatment parameter in response to determining the treatment parameter.

59. The method of any of clauses 49-58, further including: causing a motor to control a piston to administer a compressive force to a chest of the subject in response to determining the treatment parameter.

60. The method of any of clauses 49-59, further including: causing a discharge circuit to provide electrical stimulations to the subject via electrodes disposed on the subject in response to determining the treatment parameter.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1. A system, comprising:
a sensor configured to detect, during a first time period and during a second time period, a flow of blood in a blood vessel of a subject;
a display; and
a processor configured to:
determine that chest compressions are administered to the subject during the first time period;
determine that the chest compressions are paused during the second time period;
determine that the flow of blood during the second time period is greater than the flow of blood during the first time period; and
in response to determining that the flow of blood during the second time period is greater than the flow of blood during the first time period, cause the display to output a recommendation to discontinue administering the chest compressions.

2. The system of claim 1, wherein the processor is configured to determine that the chest compressions are administered to the subject during the first time period by:
identifying a chest compression artifact in data representative of the flow of blood through the blood vessel during the first time period, and
wherein the processor is configured to determine that the chest compressions are paused during the second time period by:
determining that the chest compression artifact is absent from the data representative of the flow of blood through the blood vessel during the second time period.

3. The system of claim 1, further comprising:
an additional sensor configured to detect an additional physiological parameter indicative of blood circulation in the subject,
wherein the processor is further configured to:
determine a return of spontaneous circulation (ROSC) associated with the subject by analyzing the additional physiological parameter during the second time period; and
cause the display to output an indication of the ROSC.

4. A medical device, comprising:
a sensor configured to detect a physiological parameter indicative of blood circulation in a subject during a first time period in which a treatment is being administered to the subject and to detect the physiological parameter indicative of blood circulation in the subject during a second time period in which the treatment is paused;
a processor configured to:
determine a difference between the blood circulation during the first time period and the blood circulation during the second time period by comparing the physiological parameter detected during the first time period to the physiological parameter detected during the second time period; and
determine a treatment parameter of the treatment in response to determining the difference.

5. The medical device of claim 4, wherein the sensor comprises:
an ultrasound transducer configured to:
emit an ultrasound signal toward a blood vessel of the subject; and
detect a reflection of the ultrasound signal from blood in the blood vessel of the subject, a Doppler shift between the ultrasound signal and the reflection of the ultrasound signal being indicative of a flow of blood through the blood vessel of the subject.

6. The medical device of claim 4, wherein the physiological parameter comprises a level of carbon dioxide (CO₂) in an airway in the subject, a blood oxygenation of the subject, or an electrocardiogram of the subject.

7. The medical device of claim 4, wherein determining the treatment parameter comprises:
determining a third time period to continue administration of the treatment to the subject; or
determining a fourth time period to pause administration of the treatment to the subject.

8. The medical device of claim 4, wherein the treatment comprises chest compressions, and wherein the treatment parameter comprises at least one of a frequency, a depth, or a duration of chest compressions.

9. The medical device of claim 8, further comprising:
a piston; and
a motor configured to control the piston to administer a compressive force to a chest of the subject, wherein the processor is configured to cause the motor to control the compressive force administered to the subject in response to determining the treatment parameter.

10. The medical device of claim 4, wherein the treatment parameter comprises at least one of a rate, a width, or a current of pacing pulses.

11. The medical device of claim 10, further comprising:
electrodes configured to deliver electrical stimulations to a patient via electrode leads; and
a discharge circuit, wherein the processor is configured to cause the discharge circuit to provide the electrical stimulations via the electrodes in response to determining the treatment parameter.

12. The medical device of claim 4, further comprising an output device configured to output an instruction to a user or an indication of the treatment parameter in response to determining the difference.

13. The medical device of claim 4, wherein the processor is further configured to:
determine that the treatment is administered during the first time period by identifying treatment artifacts exhibited in data representing the physiological parameter during the first time period.

14. The medical device of claim 4, wherein the processor is further configured to:
determine that the treatment is paused during the second time period by determining that a treatment artifact is absent from data representing the physiological parameter during the second time period.

15. A method, comprising:
detecting a physiological parameter indicative of blood circulation in a subject during a first time period in which a treatment is being administered to the subject;
detecting the physiological parameter indicative of blood circulation in the subject during a second time period in which the treatment is paused;
determining a difference between the blood circulation during the first time period and the blood circulation during the second time period by comparing the physiological parameter detected during the first time period to the physiological parameter detected during the second time period; and
in response to determining the difference, determining a treatment parameter of the treatment.

16. The method of claim 15, wherein detecting the physiological parameter indicative of blood circulation in the subject during the first time period in which chest compressions are being administered to the subject comprises:
determining a level of carbon dioxide (CO₂) in an airway in the subject; or
determining a level of blood oxygenation of the subject.

17. The method of claim 15, further comprising:
causing an output device to output an instruction to a user or an indication of the treatment parameter in response to determining the treatment parameter.

18. The method of claim 15, further comprising:
causing a motor to control a piston to administer a compressive force to a chest of the subject in response to determining the treatment parameter; or
causing a discharge circuit to provide electrical stimulations to the subject via electrodes disposed on the subject in response to determining the treatment parameter.

19. The method of claim 15, further comprising:
determining that the treatment is administered during the first time period by identifying treatment artifacts exhibited in data representing the physiological parameter during the first time period.

20. The method of claim 15, further comprising:
determining that the treatment is paused during the second time period by determining that a treatment artifact is absent from data representing the physiological parameter during the second time period.
